(19) United States
(12) Patent Application Publication
(10) Pub. No.: US 2025/0304943 A1
(43) Pub. Date: Oct. 2, 2025

(54) EXTENDED STABILIZATION METHOD FOR HYDROLASE ENZYMES WITH FUNCTIONAL COMPONENTS FOR BREAKING BIOPOLYMER DAMAGE IN UNDERGROUND RESERVOIR AND PIPE RELEASE

(71) Applicant: Epygen Labs FZ LLC, Dubai (AE)

(72) Inventors: Debayan GHOSH, Dubai (AE); Ineeyan ARIYARATNAM, Dubai (AE); Shrea GHOSH, Dubai (AE); Punit BANSAL, Dubai (AE); Mohammed ABDUL RAHMAN, Dubai (AE)

(73) Assignee: Epygen Labs FZ LLC, Dubai (AE)

(21) Appl. No.: 18/807,436

(22) Filed: Aug. 16, 2024

(30) Foreign Application Priority Data
Mar. 28, 2024 (IN) 202411025667

(51) Int. Cl. C12N 9/96; C09K 8/524; C12N 9/26

(52) U.S. Cl. CPC C12N 9/96; C09K 8/524; C12N 9/2414; C12Y 302/01001

(57) ABSTRACT

A method for stabilizing a hydrolase enzyme in the presence of a functional cleanup fluid component for breaking of biopolymer damage in an underground reservoir, or biopolymer damage buildup that causes friction against free movement of the drill pipe and differential stuck pipe situation. The method provides a simple way to thermally fortify and maximize the benefits of hydrolase enzymes for effective removal of biopolymer based damage from a subterranean formation. This method also has advantages over conventional treatments as it provides the capability of filter cake removal treatments as an additional application for freeing of stuck pipes diagnosed to be differentially stuck.

### CROSS-REFERENCE TO RELATED APPLICATIONS

This application claims the benefit of priority to Indian Patent Application number 202411025667, filed Mar. 28, 2024 which is hereby incorporated by reference in its entirety.

### FIELD OF THE INVENTION

The present disclosure relates to a method for stabilizing a hydrolase enzyme in the presence of a functional cleanup fluid component for breaking of biopolymer damage in an underground reservoir, or biopolymer damage buildup that causes friction against free movement of the drill pipe and differential stuck pipe situation.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Since the formation damage can be in such an area which is close to the oil producing open-hole, its presence significantly impacts production levels. Thus, effective removal of biopolymer filter cake-based damage is essential to achieve optimal production of hydrocarbons or water in water-producing wells and injectivity of such wells. Though, such biopolymer based formation damage needs to be removed to increase the flow of production fluids from the formation, it cannot be easily flushed out of the formation being nearly insoluble in aqueous fluids. Consequently, removing this filter cake often necessitates treatment with strong oxidants such as persulfates or strong acids, both of which pose considerable hazards to operational safety and the environment.

Conventionally, in efforts to revitalize under-producing wells, harsh inorganic acids like HCL were commonly used to dissolve and expediently remove damage. Nevertheless, these practices have revealed numerous drawbacks, which includes and not limited to the formation of wormholes leaking off treatment fluid, corroding tubes and plugging screens, triggering localized breakage etc. Particularly in deviated and horizontal wells, the treatment fluid often escapes through wormholes containing strong acids or oxidants, leading to contamination of gas or water layers. Such leakage diminishes the treated zone for damage restoration and compromises productivity levels.

Strong acids pose not only hazardous to handle but also corrosive to tubing and equipment, leading to sludge accumulation, crude contamination, and screen clogging, all of which incur substantial cleaning expenses. Corrosion inhibitors typically carry toxicity risks. Given that calcium carbonates frequently serve as weighting agents in drilling mud formulations, the filter cake typically comprises a blend of carbonate and biopolymer matrix. Additionally, carbonate fines produced during the drilling of carbonate rocks may also present in the filter cakes.

In addition to the above differential sticking is one of the primary causes for stuck pipe incidents during the drilling process. Once drilling mud filter cake is formed between the hole and the formation, differential sticking occurs when the pipe touches the filter cake, and the mud overbalance acts to push the pipe further into the cake, thus increasing the contact area between the pipe and the cake. Filtrate is still expelled from the filter cake between the pipe and the formation, thus shrinking the cake and allowing the pipe to penetrate further into it and so increasing the contact area still more. If the pressure difference is high enough and acts over a sufficiently large area, the pipe may become stuck.

While enzymes have been tested and used in the past to hydrolyze the biopolymer based filter cake, it is not possible to make the enzymes stable in along term formulation in presence of functional chemicals like acid precursors and organic acids. Therefore, enzyme stabilization in the presence of functionally vital formulation constituents has gained notable significance owing to the increasing number of its specialized applications. Owing to the sensitivity of the enzyme proteins to physical and chemical influences, the stabilization of enzyme formulation in the presence of critically functional ingredients for specialized application poses a big obstacle in current science. A well-known drawback of designed enzyme formulations for special use is their relatively low activity in the presence of weak organic acids.

Filter cakes form as the gel fluids are pumped into the subterranean formation and some part of the fluid leaks into the small rock pores of the formation, leaving behind the macromolecules of biopolymer gel on the rock surface, forming a relatively impermeable layer. Those biopolymers which does not form cakes, still increases viscosity on localization, which acts similar to a filtercake, blocking off production fluid. Since the filter cake is a concentrated retentate buildup of the fracturing liquid, it often contains high densities of polysaccharide. U.S. Pat. No. 5,247,995 discloses SPE Paper 21497 indicating that they can contain up to about 48% polysaccharide versus about 4% in fracturing fluids, which no doubt, is substantial.

U.S. Pat. No. 5,165,477 discloses a method of removing used drilling mud of the type comprising solid materials including at least one polymeric organic viscosifier from a well bore and portions of formations adjacent thereto comprising: injecting a well treatment fluid comprising an enzyme capable of rapidly enzymatically degrading polymeric organic viscosifier into well; and allowing enzyme to degrade polymeric organic viscosifier and well treatment fluid to disperse used drilling mud. Thus, U.S. Pat. No. 5,165,477 discloses addition of enzyme to a viscosifier.

U.S. Pat. No. 5,881,813 discloses a method for improving the effectiveness of a well treatment in subterranean formations comprising the steps of: injecting a clean-up fluid into the well wherein the clean-up fluid contains one or more enzymes in an amount sufficient to degrade polymeric viscosifiers; contacting the wellbore and formation with the clean-up fluid for a period of time sufficient to degrade polymeric viscosifiers therein; performing a treatment to remove non-polymer solids that may be present; and removing the non-polymer solids in the well to improve productivity or injectivity of the subterranean formation.

U.S. Pat. No. 5,247,995 discloses a method of increasing the flow of production fluids from a subterranean formation by removing a polysaccharide-containing filter cake formed during production operations and found within the subterranean formation which surrounds a completed well bore comprising the steps of allowing production fluids to flow from the well bore, reducing the flow of production fluids from the formation below expected flow rates and formulating an enzyme treatment by blending together an aqueous fluid and enzymes. The enzyme treatment is pumped to a desired location within the well bore and the enzyme treatment is allowed to degrade the polysaccharide-containing filter cake, whereby the filter cake can be removed from the subterranean formation to the well surface.

U.S. Pat. No. 6,110,875 discloses a method for degrading xanthan molecules comprising the step of contacting the molecules with xanthanase enzyme complex produced by a soil bacterium bearing the ATCC No. 55941 under conditions such that at least a portion of the molecules are degraded.

U.S. Pat. No. 6,936,454 discloses a composition comprising an isolated mannanase enzyme that hydrolyzes β-1,4 hemicellulolytic linkages in galactomannans at a temperature above 180° F. and that is essentially incapable of degrading the linkages at a temperature of 100° F. or less.

U.S. Pat. No. 4,617,662 discloses a method for enhancing the thermal stability of microbial alpha-amylase. The method involves adding a stabilizing amount of an amphiphile to the enzyme in its aqueous solution. Also included within the scope of the invention is the stabilized alpha-amylase formulation and its use in the liquefaction of starch.

U.S. Pat. No. 4,284,722 discloses a heat and acid stable alpha-amylase derived from an organism of the species Bacillus stearothermophilus.

U.S. Pat. No. 4,497,897 discloses a method for enhancing the shelf life during storage of protease from Subtilisin Carlsberg which involves the addition of calcium ion and a water soluble carboxylate selected from the group of formate, acetate, propionate and mixtures thereof to a solution of the enzyme.

U.S. Pat. No. 4,451,569 discloses a stable enzyme composition comprising glutathione peroxidase and at least one stabilizer compound selected from the group consisting of pentoses, hexoses, pentahydric sugar alcohols, hexahydric sugar alcohols and disaccharides.

Samborska et al., [J. Food Process Eng., 20026, 29, 287-303] report that the sucrose exhibits the largest protective effect on tested Amylase enzyme among all stabilizing compounds. The decimal reduction time of α-amylase activity increased by 33.9 times when 420 mg/mL of sucrose was added to the environment. When the same concentration of trehalose was used, the D-value increased by 6.4 times compared to the value in the buffer system. The nOH provided in the enzyme solution could not be related to the D-values for the enzyme thermal inactivation, meaning that the enzyme heat stability was not dependent on the nOH.

Lee and Timasheff [J. Biol. Chem., 1981, 256, 7193-7201] report that the results from the protein-solvent interaction study indicate that sucrose is preferentially excluded from the protein domain, increasing the free energy of the system. They report that thermodynamically this leads to protein stabilization since the unfolded state of the protein becomes thermodynamically even less favorable in the presence of sucrose.

U.S. Pat. No. 5,415,230A discloses a method and combination of materials for freeing stuck pipe involves first the spotting of a clear brine, preferably calcium chloride, calcium bromide or zinc bromide, or mixtures thereof, for a given period of time, preferably at least about 8 hours, in the stuck region of the pipe, followed by the spotting of a spotting agent selected from wetting agents, surfactants, lubricants, or mixtures thereof, in the stuck region of the pipe.

U.S. Pat. No. 8,361,938B1 discloses an aqueous or oil-based mixture containing a non-toxic, low pH, antimicrobial, acidic composition having a pH between approximately 0.5 and approximately 3.5 with and without a proppant is used as a subterranean well stimulation additive. Without a proppant, the LpHAC stimulation additive is used for acidization. In another embodiment, with a proppant, the LpHAC stimulation fluid is used in hydraulic fracturing. As a well stimulation fluid, it involves the injection of specially engineered fluids and other materials into the well bore at rates that actually cause the cracking or fracturing of the reservoir formation to create fissures or cracks in the formation to increase fluid flow of underground resources from the reservoir into the well bore.

U.S. Pat. No. 8,091,644B2 discloses nanoemulsion, macroemulsions, miniemulsions, microemulsion systems with excess oil or water or both (Winsor I, II or III phase behavior) or single phase microemulsions (Winsor IV) improve the removal of filter cakes formed during hydrocarbon reservoir wellbore drilling with OBM. Such filter cakes and their particles can contact, impact and affect the movement of the drill string undesirably resulting in a “stuck pipe” condition. The macroemulsion, nanoemulsion, miniemulsion, microemulsion systems with excess oil or water or both or single phase microemulsion removes oil and solids from the filter cake, thereby releasing the drill string from its stuck condition. In one non-limiting embodiment, the emulsion system may be formed in situ (downhole) rather than produced or prepared in advance and pumped downhole.

U.S. Pat. No. 4,614,235A discloses a formulation suitable for preparation of a spotting pill effective in the release of stuck pipe in a borehole during a drilling operation which formulation contains a mono and/or poly alkylene glycol ether and viscosified sufficiently to make the formulation compatible with a solids weighting material such as barite.

### OBJECTS OF THE INVENTION

An object of the present disclosure is to provide a method for stabilizing a hydrolase enzyme in the presence of a functional cleanup fluid component for breaking of biopolymer damage in an underground reservoir, or biopolymer damage buildup that causes friction against free movement of the drill pipe and differential stuck pipe situation.

Another object of the present disclosure is to provide a simple and effective method for fortification and stabilization of the hydrolase enzymes in the presence of an organic acids and acid precursors, at extremely high downhole temperatures of an underground reservoir, for an efficient and predictable enzymatic hydrolysis of the biopolymers embedded in the carbonate matrix of drilling mud component.

Another object of the present disclosure is to provide an enzyme and organic acid based long term stable formulation solution for freeing stuck pipes where the biopolymer-based drilling fluid cakes is in contact with the pipe and wellbore. The invention also provides a method to use this long term stable enzyme formulation to release stuck pipes where at least a major portion of the mud cake is broken by this enzyme formulation within the pipe and or the wellbore, where it aids to decrease the differential pressure across the pipe and well.

Another object of the present disclosure is to provide a novel long shelf life stable enzymatic organic acid and acid precursor formulation system for freeing stuck pipes at high downhole temperatures by its effectiveness in breaking biopolymeric muds and reducing the well fluid viscosities.

Still another object of the present disclosure is to provide a simple and effective method for the efficient removal of filter cakes from downhole conditions with relatively high temperatures, above 150° C., which could potentially deactivate most enzymes with incubation.

Still another object of the present invention is to provide a thermal fortification for the enzyme proteins for a more predictable functioning of the enzymes in hydrolyzing the biopolymers at a temperatures in the range of 60° C. to 100° C., so that biopolymer based damage can be easily removed and matrix permeability can be increased for a better production.

Yet another object of the present invention is to provide a method which is not harmful for the environment and easy to handle by the operators without any amount of hazards.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

An aspect of the present disclosure is to provide a method for stabilizing a hydrolase enzyme in the presence of a functional cleanup fluid component for breaking of biopolymer damage in an underground reservoir, or biopolymer damage buildup that causes friction against free movement of the drill pipe and differential stuck pipe situation comprising: mixing of 2 to 60% of hydrolase enzymes to the function cleanup fluid components having 1 to 45% of organic acid buffers and esters, 3 to 20% weak organic acid or acid precursors and 1 to 5% of a fatty ethoxylate and 2 to 30% of water activity reducing agents and rest being a vehicle to obtain a stable enzyme treatment fluid composition; and extending the enzyme treatment fluid composition into the underground reservoir via a wellbore with high downhole temperatures, using a drill string or deployed using a coiled tube or bullheaded into the fractures, while injecting the enzyme treatment fluid composition below or above the fracture pressure and shut off the contents for a period of time, or below or above the stuck pipe region where the drill pipe is differentially stuck, allowing to react and disintegrate the biopolymer based formation damage or filter cake or biopolymer caused stuck causing material completely and subsequently removed by normal flushing techniques in a known manner, wherein the higher stability of the hydrolase enzyme of the enzyme treatment fluid composition is achieved despite the presence of the function cleanup fluid components, rendering a unique formulation that results in the stability of the enzyme protein without salting out or precipitation of the protein in presence of pH lowering organic acid, wherein the enzyme treatment fluid composition disintegrates partially or completely the biopolymer filtercake by virtue of hydrolyzing the cross linking and matrix components of the biopolymer, also providing a reduction in the amount of force required to free said pipe.

Various objects, features, aspects and advantages of the inventive subject matter will become more apparent from the following detailed description of preferred embodiments.

### BRIEF DESCRIPTION OF THE DRAWINGS

The following drawings form part of the present specification and are included to further illustrate aspects of the present invention. The disclosure may be better understood by reference to the drawings in combination with the detailed description of the specific embodiments presented herein.

FIG. 1 illustrates effect of weak organic acids on the amylase enzyme activity.

FIG. 2A illustrates standard mudcake without treatment.

FIG. 2B illustrates mudcake removal after treatment.

FIG. 3 shows stuck pipe illustration in the well having pipe (1), wellbore (2), mudcake (3), drilling mud in wellbore (4) and pressure causing stuck (5).

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it is individually recited herein.

All processes described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present invention can be implemented in numerous ways, including as a system, a method or a device. In this specification, these implementations, or any other form that the invention may take, may be referred to as processes. In general, the order of the steps of the disclosed processes may be altered within the scope of the invention.

Various terms as used herein are shown below. To the extent a term used in a claim is not defined below, it should be given the broadest definition persons in the pertinent art have given that term as reflected in printed publications and issued patents at the time of filing.

Once subjected to high temperatures that could otherwise deactivate enzymes in downhole conditions, effectiveness of a stabilized filter cake breaking hydrolase enzyme system can be established in the laboratory. This can be done, either by checking the increase of filtration rate through a biopolymer based filter-bed of similar composition prepared and cured in the lab or simply by estimating the reducing sugar produced as a hydrolysis product of the biopolymer in the bed. It has been reported that although enzymes potentially offer a number of advantages over conventional chemical catalysts, they are generally unstable in extreme conditions and they get deactivated rapidly by heat and other environmental modifications such as changes in pH and ionic disbalance. Since the active site of the enzyme consists of amino acids brought together only in the native three-dimensional structure, an unfolded enzyme loses its catalytic activity.

Therefore, fortifying and protecting the hydrolase enzymes to co-exist with the breaking fluid system based functional chemicals e.g. organic acids and acid precursors, to deliver an efficient enzyme hydrolysis at extremely high downhole temperatures not only bears commercial value of enzyme dosage in treatment fluid formulation, it also provides higher predictability of the enzyme kinetic rate in downhole conditions, bringing about ease in operation, planning and calculation.

The long term formulation stability of enzyme formulations containing weak organic acid and acid precursors has not been discussed in any prior art, where a particular difficulty has been the rapid decrease of enzyme activity in the formulation during normal storage conditions in the presence weak organic acid or precursors, which perturbs the pH condition and decreases the functionality of enzyme activity during oil and gas operations. The stability of such enzyme in presence of a weak acid has been a problem area.

The present disclosure relates to a method for long term stabilizing of a hydrolase enzyme in presence of functional cleanup fluid components for breaking of biopolymer damage in oil and gas wells including weak organic acids, which provides one step solution utilized for biopolymer-based mud filter-cake clean-up operation for new and old drilled wells and dissolution of biopolymer filer-cake build-up that produces friction against free movement of drill pipes, causing a stuck pipe situation. For horizontal wells drilled with water-based mud system, the bio-polymeric drilling fluid form a strong and nearly impermeable mud cake coating on the wellbore wall, removal of which is of utmost importance to restore the productivity or injectivity of the well. An enzyme and a weak organic acid system attack the bio-polymers and carbonate bridging material of the cake, resulting in degradation of the filtercake.

The key invention highlights a stable formulation for a wide range of operations involving and not limited to the removal of polymer-based drilling fluid caused filter cake in the oil and water wells and removal of biopolymer filter cake build-up that produces friction against free movement of drill pipes, causing a stuck pipe situation (Stuck-Pipe), along with saving field operation time providing flexible operational solutions for a variety of services required in the well. While enzymatic treatment is considered an effective and safe solution to tools, conventional enzyme formulations are not stable in presence of functional chemicals, which is required to be added at the site of application. The present disclosure provides technique for stabilizing a wide range of enzymes in presence of organic acids, acid precursors and buffers in such a way that one drum formulation can be stored for long periods as long as 12 to 24 months and pumped as convenient for completing the filter cake breaking or stuck pipe removal job for oil wells.

The present disclosure relates to conformational stability of enzyme protein and in particular, to method in preserving the long-term stability of enzymatic activity in enzyme formulations in the presence of weak organic acid, buffers, acid precursors used in drilling mud cake removal and to demonstrate the function of a specially developed enzyme protein system that can release stuck pipes withstanding harsh downhole conditions. Therefore, a long term stable hydrolase enzyme formulation in presence of the components of the breaking fluid system based functional chemicals e.g. organic acids and acid precursors not only bears commercial value in terms of enzyme carrying to drilling sites and economic dosages in treatment fluid formulation, it also provides higher predictability of the enzyme's kinetic rate in downhole conditions.

An embodiment of the present disclosure provides a method for stabilizing a hydrolase enzyme in the presence of a functional cleanup fluid component for breaking of biopolymer damage in an underground reservoir, or biopolymer damage buildup that causes friction against free movement of the drill pipe and differential stuck pipe situation comprising: mixing of 2 to 60% of hydrolase enzymes to the function cleanup fluid components having 1 to 45% of organic acid buffers and esters, 3 to 20% weak organic acid or acid precursors and 1 to 5% of a fatty ethoxylate and 2 to 30% of water activity reducing agents and rest being a vehicle to obtain a stable enzyme treatment fluid composition; and extending the enzyme treatment fluid composition into the underground reservoir via a wellbore with high downhole temperatures, using a drill string or deployed using a coiled tube or bullheaded into the fractures, while injecting the enzyme treatment fluid composition below or above the fracture pressure and shut off the contents for a period of time, or below or above the stuck pipe region where the drill pipe is differentially stuck, allowing to react and disintegrate the biopolymer based formation damage or filter cake or biopolymer caused stuck causing material completely and subsequently removed by normal flushing techniques in a known manner, wherein the higher stability of the hydrolase enzyme of the enzyme treatment fluid composition is achieved despite the presence of the function cleanup fluid components, rendering a unique formulation that results in the stability of the enzyme protein without salting out or precipitation of the protein in presence of pH lowering organic acid, wherein the enzyme treatment fluid composition disintegrates partially or completely the biopolymer filtercake by virtue of hydrolyzing the cross linking and matrix components of the biopolymer, also providing a reduction in the amount of force required to free said pipe.

In an embodiment, the underground reservoir is a hydrocarbon reservoir or water reservoir. The hydrocarbon is oil and/or gas.

In an embodiment, the hydrolase enzyme is selected from a group consisting of an alpha amylases, beta amylases, gamma amylases, cellulases, endo cellulases, exo cellulases, cellobiase, beta-glucanase, hemicellulase, mannanase, galactomannanase and combination thereof.

In an embodiment, two or more hydrolase enzyme are mixed together based on the nature of biopolymer used in mud formulation.

In an embodiment, the hydrolase enzymes are mixed in the same ratio of the biopolymer substrates used in the mud formulation, on basis of declared enzyme activity units.

In an embodiment, the hydrolase enzyme includes a hydrolase enzyme mix of amylases, xanthanases, cellulose enzyme and/or hemicellulase-mannanase, endoglycanases, cellobiohydrolase enzymes.

In an embodiment, the alpha amylase hydrolase enzyme that catalyses the endo-hydrolysis of 1,4-alpha-glycosidic linkages in starch, glycogen, and related polysaccharides and oligosaccharides containing 3 or more 1,4-alpha-linked-glucose units.

In an embodiment, the water activity reducing agent is a selected from a group consisting of sucrose, sorbitol, mannitol, glycerol, trehalose, propylene glycol and combination thereof. Preferably, the water activity reducing agent is a selected from sucrose, mannitol and glycerol. More preferably, the water activity reducing agent is sucrose.

In an embodiment, the weak organic acid is selected from a group consisting of acetic acid, formic acid, citric acid, lactic acid and combination thereof. Preferably, the weak organic acid is selected from acetic acid, formic acid and citric acid. More preferably, the weak acid is acetic acid.

In an embodiment, the acid precursor is an ester of acetic acid, ester of formic acid, ester of citric acid, ester of lactic acid or a combination thereof. Preferably, the acid precursor is selected from ester of acetic acid, ester of formic acid and ester of citric acid. More preferably, the acid precursor is ester of acetic acid.

In an embodiment, the organic acid buffers and ester is selected from a group consisting of chloride, carbonates, sulphate, nitrate buffers and ethanoates esters. The organic acid buffers and ester is selected from a group consisting of calcium chloride, sodium chloride, sodium ethanoate and combination thereof. Preferably, the organic acid buffers and ester is selected from calcium chloride, sodium chloride, sodium ethanoate. More preferably, the organic acid buffers and ester is selected from calcium chloride and sodium ethanoate.

In an embodiment, the fatty ethoxylate is selected from a group consisting of alkylphenol ethoxylates, alcohol ethoxylates, amine ethoxylates, acid ethoxylates, castor oil ethoxylates, ester ethoxylates and combination thereof. The fatty alcohol ethoxylate is selected from a group consisting of lauryl alcohol ethoxylate, cetyl alcohol ethoxylate, strearyl alcohol ethoxylate, oleyl alcohol ethoxylate, tridecyl alcohol ethoxylate and combination thereof.

In an embodiment, the hydrolase enzymes are a mix and are liquid enzyme formulations.

In an embodiment, the vehicle is selected from a group consisting of water, brine water and sea water.

In an embodiment, the biopolymer is selected from a group consisting of starch, xanthan, cellulose, guar derivatives and combination thereof.

In an embodiment, the temperature of the formation bearing the biopolymer based filtercake or mud damage of the reservoir is at least 60° C. or higher.

In an embodiment, the enzyme treatment fluid composition is shut off in the reservoir for at least 45 minutes.

In an embodiment, the wellbore is vertical, deviated inclined or horizontal.

Another embodiment of the present disclosure provides a stable enzyme treatment fluid composition comprising: 2 to 60% of hydrolase enzymes; 2 to 30% of water activity reducing agents; 1 to 45% of organic acid buffers and esters; 3 to 20% weak organic acid or acid precursors; 1 to 5% of a fatty ethoxylate and rest being a vehicle.

In an embodiment, the composition further comprises an additive. The additive is selected from a group consisting of chelating agent, antifoam or biocide and combination thereof.

In an embodiment, the present disclosure provides a method of stabilizing hydrolase enzyme formulations as a single product along with other functional ingredients of the treatment fluid like acid precursors and organic acids, rendering it stable for long term in a single drum, for further pumping the same into a subterranean formation bearing high downhole temperatures and harsh chemical and physical conditions, for effective removal of biopolymer based filter cake and biopolymer filter cake build-up that produces friction against free movement of drill pipes, causing a stuck pipe situation, without the enzyme getting de-activated, which prior to this invention was not possible to make a stable formulation for several months shelf life in presence of the enzymes and functional drilling treatment chemicals. Possibility of obtaining a long shelf life single enzymatic product formulation that when pumped downhole efficiently removes filter cake and increases the permeability of the formation or the fracture, stimulating the well to produce at a higher rate and also releases stuck pipes during drilling operations, which is otherwise a major operational challenge, that is resolved by the present invention. The key invention highlights the one formulation stable solution for a range of filtercake removal operations involving removal of polymer-based drilling fluids in the oil and water wells and resolving stuck pipes caused by biopolymer buildup.

The method relates to hydrolase enzyme chosen based on the nature of the drilling fluid biopolymer substrate, e.g. range of amylases and xanthanases for starch xanthan substrates, range of cellulases for a PAC, HEC and CMC substrates or range of mannanases for guar derivative based substrates, to long term stabilize the enzymes in presence of functional cleanup fluid components and ensure that it works at an efficient kinetic rate, even when subjected to high downhole temperatures. The scope of the present disclosure particularly addresses oil, gas or water producing wells with high downhole temperatures ranging between 80 to 150° C., that is 176 to 302° F.

In an embodiment, the present disclosure provides a method for protein stabilizers and sucrose based stabilization of enzymes for breaking of biopolymer damage in gas and oil wells, said enzyme includes amylases, xanthanases and/or a biopolymer hydrolase enzymes mix of cellulase and/or hemicellulase-mannanase, endoglucanases, cellobiohydrolase enzymes, the said method comprises step of preparing long term stable enzyme treatment fluid composition by combining the hydrolase enzymes with water activity reducing agents, protein stabilizers and polyols and surfactant ethoxylates, which formulation can be stored in normal storage condition, and further extending into an oil or gas or water producing reservoir via a wellbore with high downhole temperatures, using a drill string or deployed using a coiled tube or bullhead into the fractures, while injecting the treatment fluid below or above the fracture pressure and shut off the contents for a period of time, allowing to react and disintegrate the biopolymer based formation damage or filter cake or biopolymer buildup causing stuck pipe completely and subsequently removed by normal flushing techniques known manner.

In an embodiment, the present disclosure also provides a method to use the long term stable enzyme formulation to release stuck pipes where at least a major portion of the mud cake is broken by this enzyme formulation within the pipe and or the wellbore, where it aids to decrease the differential pressure across the pipe and well.

In an embodiment, the present disclosure provides a method for treating an underground reservoir, which method comprises introducing into the reservoir a treatment fluid comprising, dissolved or dispersed in water, a hydrolase enzyme or a combination of hydrolase enzymes aimed at a complex biopolymer substrate, and a stabilizer system to protect the hydrolase enzyme from degradation caused by organic acids and esters used in formulation for functional reasons and thermal deactivation in high downhole temperatures.

In an embodiment, the method of the present disclosure fortifies and stabilizes hydrolase enzymes, enabling these to stay active for long term in formulation in presence of the functional breaking fluid system based functional chemicals e.g. organic acids and acid precursors and remains in a condition to function at high downhole temperatures when pumped into the well and partially or completely disintegrate biopolymer component of filter cake and carbonate matrix of the cake formed due to using polysaccharide-containing drilling fluids, in subterranean formations. Without suitable stabilization or fortification, the hydrolase enzymes would be completely or partially deactivated in the treatment fluid, and not perform as predicted, in an efficient manner.

When utilizing a biopolymeric viscosifier-based drilling mud in a subterranean reservoir, a filter cake forms on the rock matrix as the aqueous fluid from the polymer suspension is filtered through the small rock pores. This filter cake contributes to decreased permeability of the formation and slows down production substantially. This filter cake of biopolymeric deposits consists primarily of the long chain polysaccharide biopolymers embedding the carbonate weighting agents, often used in water based mud. These polysaccharides may include starch or amylum, xanthan gum and its derivatives, cellulose and its derivatives like carboxymethyl cellulose, or guar derivatives such as hydroxymethyl guar or hydroxypropyl guar.

Starch-based biopolymeric viscosifiers in formation damage consist of glucose polymers linked together by the alpha-1,4 and alpha-1,6 glucosidic bonds. Due to presence of two types of linkages, the alpha-1,4 and the alpha-1.6, different conformational existence are possible for starch molecules. An unbranched polymer with only alpha-1,4 glucosidic bonds, containing 500 to 2000 glucose subunits, is termed amylose. In contrast, the presence of alpha-1,6 glucosidic linkages results in a branched glucose polymer known as amylopectin. The degree of branching in amylopectin occurs approximately once per every twenty-five glucose units in the unbranched segments.

Another biopolymer employed as a mud viscosifier is cellulose, which is the most abundant biopolymer found on earth. Cellulose consists of D-glucopyranose monomer units connected by β (1-·4) glycosidic linkages. The cellulose molecule forms a linear, almost extended chain with a two-fold screw axis, where successive glucose residues rotate 180 degrees relative to each other, and the glycosidic oxygens point alternatively up and down.

Still another, the biopolymer-based gelling agent Guar gum, also known as Galactomannan, is a high molecular weight carbohydrate polymer derived from the seeds of the guar plant (Cyampopistetragonolobus). The seed comprises three main parts: the hull (14-17%), the endosperm (35-42%), and the germ (43-47%). Guar gum is a polysaccharide where galactose units are randomly distributed along the mannose backbone, with an average ratio of 1:2 of galactose to mannose.

While enzymes present several benefits compared to traditional chemical catalysts, they typically exhibit instability under extreme conditions and are quickly deactivated by heat and other environmental factors like pH alterations and ionic imbalances. As the active site of an enzyme relies on the specific arrangement of amino acids within its native three-dimensional structure, denaturation of the enzyme results in the loss of its catalytic activity.

Generally, a disadvantage of most commercial enzyme formulations is their long term stability in addition to weak organic acid and acid precursors. They generally degrade in extreme conditions and are inactivated rapidly by pH drop and heat and other environmental modifications that result in electrostatic interactions between charged amino acids. These electrostatic interactions are dependent on pKa values of amino acid side chains, influence ionization equilibria of acidic and basic groups and alter their pKa values, which play an important role in defining the pH-dependent characteristics of enzyme stability. Enzymes being amphoteric molecules have a number of discrete benefits over conventional chemical catalysts. They contain a large number of acids as well as basic groups on their surface. Acid dissociation constant also known as acidity constant or acid-ionization constant (pKa) and pH play a crucial role in enzyme stability. The charges on enzymes differs, depending on their pKa and the pH of their environment. This affects the total net charge of the enzymes, distribution of charge on their exterior surfaces and also the reactivity of the catalytically active groups. Taken together, the changes in pH alter the activity, structural stability and solubility of the enzyme.

The method in this invention includes an addition of water activity reducing agents to aqueous solutions of the hydrolase enzymes in the treatment fluid formulation, thus strengthening the hydrophobic interactions among nonpolar amino acid residues. These interactions, together with hydrogen bonds and ionic and van der Waals interactions, are essential to maintain the native, catalytically active structure of the enzyme. Thus, the strengthened hydrophobic interactions make protein macromolecules more rigid, and therefore more resistant to thermal unfolding, which would otherwise render the Hydrolase enzymes inactive in downhole conditions, when the treatment fluid is pumped and shut in for hours at extremely high temperatures.

In the particular method, the water activity reducing agents is sucrose, a disaccharide of glucose and fructose with an a (alpha) 1,2 Glycosidic linkage and with molecular formula C₁₂H₂₂O₁₁ The concentration of sucrose in the treatment fluid should be sufficient to attain thermal stability of the 3-D structure of the enzyme protein in high downhole temperatures. The concentration of water activity reducing agents incorporated into the treatment fluid of the present invention will be from 2% w/v but may be up to 60% w/v (1 to 50 kgs per m³). In general it has been found that sucrose when used in combination with the hydrolase enzymes and 2% brine or sea water, substantially fortifies the enzymes against harsh downhole conditions and organic salt buffers, such as calcium chloride, sodium chloride, sodium ethanoate (at least 1% to 45% w/w or higher), as explained in more detail below.

This technique of stabilizing enzymes in the treatment fluid formulation, utilizing water activity reducing agents in aqueous media, does not alter the protein conformation. Instead, it affects the physicochemical properties of the system, such as the solvent structure, leading to protein stabilization. The solvent composition in the immediate domain of protein differs from that of the bulk solvent, and the difference is a function of the concentration of the co-solvent. This co-solvent, when added to the enzyme's aqueous solution, is excluded from the protein domain, thereby increasing the free energy shifting the thermodynamic equilibrium towards the native state.

The unique multienzyme preparation has been formulated and stabilized in the presence of a range of functional components, including and not limited to organic acids and their corresponding buffers and esters. A suitable enzyme for the composition comprising multiple enzymes including hydrolases and lyases, which has been formulated with weak organic acids and buffers and esters. Generally, a disadvantage of most commercially available enzyme is its lack of stability on addition to weak organic acids or buffers. They degrade in non-ideal pH conditions and are inactivated rapidly by heat and other environmental factors e.g. salinity etc. Changes in pH result in electrostatic interactions between charged amino acids throwing them out of functional conformity. These electrostatic interactions are dependent on pKa values of amino acid side chains and are influenced by ionization equilibria of acidic and basic groups that alter their pKa values. This plays an important role in defining the pH-dependent characteristics of enzyme stability.

When the hydrolytic activity of a biopolymer degrading enzymes are stabilized for long-term storage in the presence of functional components, as per the present method, gelatinized and polymerized starch, along with other biopolymers present in the formation damage, are actively attacked by the enzyme components of the treatment fluid upon contact. Depending on the position of the bond being attacked relative to the chain's end, the resulting water-soluble products may include dextrin, maltotriose, maltose, glucose, and so forth.

As an illustration of biopolymer hydrolysis, the bond specificity attacked by alpha-amylases varies depending on the enzyme's source, whether bacterial or fungal. Typically, bacterial alpha-amylase indiscriminately cleaves only the alpha-1,4 bonds, effectively liquefying the gelatinized starch present in the filter cake of the damage. This process initially reduces viscosity by cutting down chain lengths and ultimately renders the starch soluble in water.

Within the major categories of Amylase enzymes, the long-term stabilization approach in the presence of treatment fluid chemicals pertains to Alpha or Endo Amylases, which are 1,4-α-D-glucan glucanohydrolase. The stabilized Endo-Amylase, by acting at random locations along the starch chain of the formation damage in contact, will break down long-chain carbohydrates ultimately yielding maltotriose and maltose from amylose, or maltose, glucose and “limit dextrin” from amylopectin. Due to its ability to act at various locations on the substrate, α-amylase exhibits a faster rate of action compared to β-amylase of Glucoamylases, making it the preferred enzyme for this method.

Depending on the type of starch present in the formation damage, Beta or Exo Amylases, 1,4-α-D-glucan maltohydrolase, function from the non-reducing end to catalyze the hydrolysis of the second α-1,4 glycosidic bond, resulting in the cleavage of two glucose units (maltose) at a time. Finally, Amyloglucosidase or glycosidic linkages and the last α (1-4) glycosidic linkages at the non-reducing end of amylose and amylopectin, yielding glucose.

Another instance of biopolymer hydrolysis involves the long-term stabilization of cellulolytic hydrolase enzymes, or cellulases, to effectively operate under harsh downhole conditions of temperature and chemistry. These enzymes catalyze the hydrolysis of 1,4-glycosidic linkages within cellulose biopolymers such as PAC or HEC present in the formation damage. The process employs three distinct cellulolytic activities: Exoglucanases, also known as cellobiohydrolases, hydrolyze cellulose from the free chain ends, primarily yielding cellobiose. Endoglucanases, in contrast, randomly cleave internal linkages within the cellulose chain. Finally, β-Glucosidases catalyze the hydrolysis of small oligomers, predominantly trimers and dimers, into water-soluble monomers.

An additional illustration of biopolymer hydrolysis involves the long-term stabilization of Guar derivative biopolymer hydrolase enzymes to effectively function under harsh downhole conditions, encompassing extreme temperatures and chemical environments. These enzymes, such as Galactomannanase or Mannanase types of Hemicellulase, target the galactomannosidic and mannosidic linkages within the guar residue, thereby cleaving the molecules into soluble monosaccharide and disaccharide fragments. This process renders the mud damage easily removable. Specifically, these Hydrolase enzymes catalyze the hydrolysis of (1,6)-alpha-D-galactomannosidic and (1,4)-beta-D-mannoside linkages between the monosaccharide units in the guar-containing filter cake, respectively.

In the particular method, sufficient amounts of active and stable hydrolase enzyme should be present in the enzyme treatment fluid composition, to be able to efficiently disintegrate and slacken the rigid filter Biopolymer matrix of the formation damage and finally remove the filter cake or slacken the differentially stuck pipe. Depending on the type of Biopolymer used in mud formulation, an Amylase, a Cellulase or a Hemicellulase enzyme should be chosen to work independently or in a combination, whereas the combination of enzymes should be in the same ratio of the biopolymer substrate used in mud formulation. Typically a single enzyme or multiple enzymes together of total 2 to 60% w/v or, 1 to 50 kg/m³ of the enzyme treatment fluid composition has been found to be suitable for an effective dissolution of the Biopolymer Matrix when Thermally fortified as per this method.

As per the method, the stable enzyme treatment fluid composition is prepared by dissolving the water activity reducing agents (thermal stabilizer i.e. sucrose) in suitable water, like city water or produced brine water or sea water and finally the hydrolase enzymes are added to the mixing tank. Since no harsh chemicals are used in this method, tank material can be mild steel or a polymer like HDPE. Subsequently other chemical additives like fatty ethoxylate, organic acid buffers and esters and weak organic acid or acid precursors are added optionally other additives such as chelating agents, antifoam or biocides can be added as are commonly used in the oil industry. A suitable biocide controls growth of unwanted microbes in the treatment fluid during pumping and shut in hours.

The mixed fluid is injected into the subterranean formation through injection or production wells. For newly drilled wells, the fluid can be introduced via the drill string using mud pumps or by employing coiled tubing or bullheading techniques to contact the zone of formation damage or filter cakes.

Based on the formation and reservoir properties, the treatment fluid is introduced at pressures either below or above the fracture pressure. For open hole or near wellbore treatments, the volume of treatment fluid typically ranges from 120% to 200% of the open hole or wellbore volume, considering factors such as leak-offs and dead volumes.

Since enzyme kinetics are directly influenced by pH and temperature conditions at downhole, the enzyme treatment is shut in the formation for a sufficient duration of time to effectively degrade the damage. According to this method, the shut-in period typically ranges from 45 minutes to a week, and preferably for a time period in the range of 3 to 36 hours for efficient damage removal.

The present method is designed to offer a single step solution for stuck pipe challenges during drilling and workover periods of the well. The key invention delivers a stable enzymatic solution involving breakdown of various polymer-based drilling fluids at high downhole conditions that help to release stuck pipes caused by drilling mud buildup.

While the foregoing describes various embodiments of the disclosure, other and further embodiments of the disclosure may be devised without departing from the basic scope thereof. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention.

The thermal stabilizing effect as per the present disclosure was tested by dual method of filtration rate increase and reducing sugar production for a range of treatment fluids with different concentrations of thermally stabilizing sugar. The different stable enzyme treatment fluid composition is given in Table 1.

Blank filtration rate through perforated disc with Whatman No 1 Filter-paper was checked at 2 Atmosphere Vacuum using 2% brine solution. The Disc was then damaged by passing mud containing Biopolymers like Starch, Xanthan gum, Calcium Carbonate weighting agents and Sodium Hydroxide under 2 Atmosphere vacuum. The filter cake was allowed to settle further by incubating at 90° C. for 4 hours. The filter cake was then incubated with different enzyme treatment fluid composition at 105° C. for 12 hours. After incubation, the filtercakes were subjected to 2% brine flow at 2 atmosphere and Filtration rate tested and presented in Table 2. The Residual filter cake and Filtration Permeates of each sample was collected separately & mixed well. Reducing sugar production due to Enzymatic Hydrolysis was estimated by the following method. The aliquoted samples (1.5 ml) were centrifuged for 15 min at 10,000 rpm. The supernatant was collected and centrifuged for 15 min at 10,000 rpm. The pellets were discarded and 700 pL supernatant was taken from all the tubes in Boiling Tubes. To this, 50 mL Sodium acetate buffer (pH 4.8) and 1.2 mL DNS (Dinitrosalicylic Acid Solution) were added and tubes were incubated for 30 min at room temperature. Subsequently, the tubes were put in Boiling water Bath and incubated at 95° C. for 15 mins. The tubes were then transferred to a water bath at Room Temperature for 10 min and then incubated at Room Temp for 10 min. The coagulated samples were then crushed well in their own contents and fully collected in Eppendorf tubes and centrifuged at 10,000 rpm for 10 min. Supernatant was collected and diluted with DM water (as per below table 3) to get sufficient volume for Cuvette used in Spectrophotometer. Absorbance was measured at 540 nm and calculated back using the formula: Final O.D of sample=[(Vol of Supernatant+Vol of diluting water)/Vol of Supernatant]*Measured O.D. The Reducing sugar production was observed as per Table 3.

TABLE 1
Enzyme treatment fluid compositions.

| | | Weak | Water | Organic Acid | Fatty | |
|---|---|---|---|---|---|---|
| | Hydrolase | Organic | activity | Buffers and | Ethoxylates | |
| | Enzymes | Acid | Reducing | Esters | (lauryl | |
| | (alpha | (acetic | agent | (sodium | alcohol | |
| Description | amylase) | acid) | (Sucrose) | ethanoate) | ethoxylate) | DM Water |
| Control | 40% | 7% | Nil | Nil | Nil | Remaining |
| Formulation 1 | 40% | 7% | 30% | 10% | 1% | Remaining |
| (TFA1) | | | | | | |
| Formulation 2 | 40% | 7% | 30% | 7% | 1% | Remaining |
| (TFA2) | | | | | | |
| Formulation 3 | 40% | 7% | 25% | 5% | 1% | Remaining |
| (TFA3) | | | | | | |
| Formulation 4 | 40% | 7% | 20% | 2% | Nil | Remaining |
| (TFA4) | | | | | | |

TABLE 2
Filtration rate

| | | Brine Flow as % | |
|---|---|---|---|
| | Brine Flow Rate | of Blank Enzyme | Crushability of |
| Sample | through Damage | Treatment | Filter Cake |
| Mud Control | 0 | 4% | Hard cake |
| Mud + TFA1 | 0 | 96% | Easily crushable |
| | | | and largely |
| | | | soluble cake |
| Mud + TFA2 | 0 | 82% | Easily crushable |
| | | | and largely |
| | | | soluble cake |
| Mud + TFA3 | 0 | 69% | Crushable cake |
| Mud + TFA4 | 0 | 58% | Moderately |
| | | | crushable cake |

TABLE 3
Reducing sugar production

| | Dilution | | | Blanked OD |
|---|---|---|---|---|
| | Supernatant | Water | | (Final OD − |
| Sample | (μl) | (Ml) | Final OD | Mud Control OD) |
| Mud Control | 200 | 3 | 0.079 | 0 |
| Mud + TFA1 | 200 | 3 | 0.991 | 0.831 |
| Mud + TFA2 | 200 | 3 | 0.626 | 0.547 |
| Mud + TFA3 | 200 | 3 | 0.430 | 0.351 |
| Mud + TFA4 | 200 | 3 | 0.390 | 0.311 |

### Amylase Assay

Methodology of amylase assay: In numbered tubes, 5 maltose dilutions ranging from 1 mM to 10 mM were prepared. Distilled water was used as a blank. Into a series of corresponding numbered tubes, pipette 1 ml of each dilution of maltose. Added 1.2 ml 3,5-dinitrosalicylic acid (DNS) solution to each tube. Let it stand at room temperature for 15 min. Incubated at 95° C. in a water bath for 5 min and cooled in a room temperature (about) 20-35° water bath for 10 min. Transferred to a test tube stand and incubated for 10 min at room temperature. 200 μl of the reaction mixture was taken and diluted with 3 ml distilled water in a cuvette. Mixed and read at 540 nm.

Assay Procedure: Equilibrate 0.50 ml of the 1.2% starch substrate at 90° C. in covered glass test tubes for 10 min each. At exactly 10 min, at timed intervals of 20 seconds (start timer), rapidly 0.5 ml of the prepared enzyme dilution was added to the equilibrated starch substrate. Recap, vortex and incubated at 90° C. for exactly 10 min. Twenty (20) seconds later, prepare the second tube and then continue in the same manner with the rest of the tubes.

After exactly 10 min, add 1.2 ml of DNS-solution, recap and vortex. Allow this solution to stand at room temperature for 15 min. Cover the tubes with rubber stoppers or plastic caps to prevent evaporation. Incubate for 5 min in a water bath at 95° C. with respective controls. After boiling, cool down the samples for 10 min in a water bath at room temperature, then let sit at room temperature (about 20-35° C.) for 10 min. Measuring the absorbance of the enzyme samples at 540 nm against distilled water blank and rate of the appearance of reducing sugar is a measure of amylase activity.

Run one enzyme blank for each sample in the assay. Incubate 0.5 ml of starch substrate at 90° C. for 10 min in glass test tubes. Add 1.2 ml of DNS solution and 0.5 ml of a suitable enzyme dilution (same as above). Allow this solution to stand at room temperature for 15 min. Cover tubes to prevent evaporation. Boil for 5 min in a boiling bath. Measure the absorbance of the enzyme blanks at 540 nm against distilled water blank.

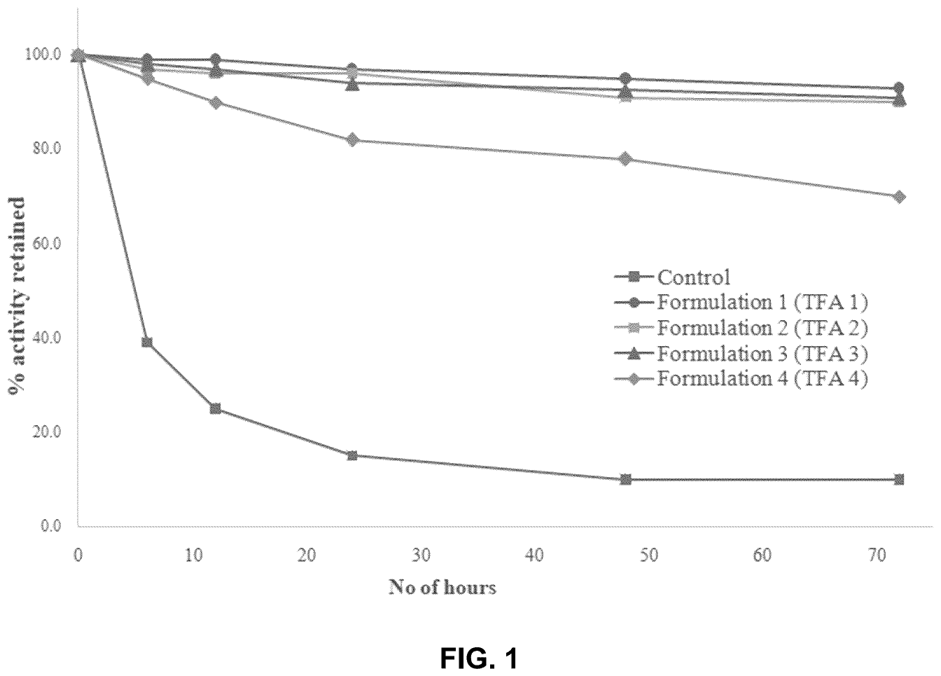

FIG. 1 showed the effect of weak organic acid on the amylase enzyme activity. Control graph showed that the enzyme activity was decreased in the presence of weak organic acid indicating poor stability of enzyme. Whereas, the formulations TFA1 to TFA3 retain the enzyme activity as shown in the FIG. 1 and Table 4 indicating that formulations TFA1 to TFA3 showed long term stability of enzyme. Stabilizing effects mostly coming from the water activity reducing agent present in the formulation. The stabilizing system saves the enzyme protein and retains the enzyme activity. Further, the enzyme activity of TFA4 also decreased due to absence of ethoxylates. This indicates that the components of formulations (TFA1 to TFA3) in desired amount able to retain the enzyme activity.

TABLE 4
Stability of enzyme.
% Activity Retained

| | | Formu- | Formu- | Formu- | Formu- |
|---|---|---|---|---|---|
| No. of | | lation 1 | lation 2 | lation 3 | lation 4 |
| Hours | Control | (TFA1) | (TFA2) | (TFA3) | (TFA4) |
| 0 | 100.0 | 100.0 | 100.0 | 100.0 | 100.0 |
| 6 | 39.0 | 99.0 | 97.0 | 98.0 | 95.0 |
| 12 | 25.0 | 99.0 | 96.0 | 97.0 | 90.0 |
| 24 | 15.0 | 97.0 | 96.0 | 94.0 | 82.0 |
| 48 | 10.0 | 95.0 | 91.0 | 92.5 | 78.0 |
| 72 | 10.0 | 93.0 | 90.0 | 91.0 | 70.0 |

### Differentially Stuck Pipe Release Estimation by Stabilized Hydrolase Enzymes

The HPHT test method was designed to evaluate and optimize treatments for removing polymeric muds and filter cake created by fluids used during well drilling. The equipment used for the test is a HPHT filter loss apparatus for evaluating API filter loss in drilling fluid at high-temperature and high-pressure conditions. In this test, reservoir temperature was created with the help of a heating chamber, a homogeneous porous ceramic disc was used to represent porous reservoir rock, and pressure is applied to mimic well overbalance pressure. Biopolymeric Mud formulation used in the HPHT unit is shown in Table 5.

The stable enzyme treatment fluid compositions (Table 1), especially designed to address the stuck-causing biopolymer mud etc. to study the hydrolysis/breakdown of these components collectively. The rate of biopolymer breakdown by enzyme in the HPHT filtration unit was evaluated. HPHT test was carried out in the laboratory before application in the field for stuck pipe removal.

TABLE 5
Biopolymeric Mud formulation used in the HPHT unit

| Ingredients | Amount | |
|---|---|---|
| Xanthan | 4 | g |
| Starch | 20 | g |
| CaCO₃ (Med) | 30 | g |
| CaCO₃ (Fine) | 30 | g |
| CaCO₃ (Large) | 30 | g |
| PAC (L) | 14 | g |
| PAC(R) | 3 | g |
| NaCl | 250 | g |

| | |
|---|---|
| Soda Ash | 1.4 |
| pH of mud | 9-9.5 |

Biopolymer breakdown by enzyme treatment fluid compositions and percentage clean up is given in Table 6. Results in Table 6 showed that TFA1 to TFA 3 of the present invention showed higher clean up as compared to control and TFA4. TFA4 is showing much less range of cleanup. This indicate that the present formulation TFA1 to TFA 3 not only break the mudcake but also able to suck pipe removal efficiently.

TABLE 6
Biopolymer breakdown by enzyme treatment fluid compositions.

| Description | % clean up |
|---|---|
| Control | Nil |
| TFA1 | 92% to 98% |
| TFA2 | 88% to 92% |
| TFA3 | 85% to 90% |
| TFA4 | 80% to 85% |

### Field Application Case History (Stuck Pipe Release):

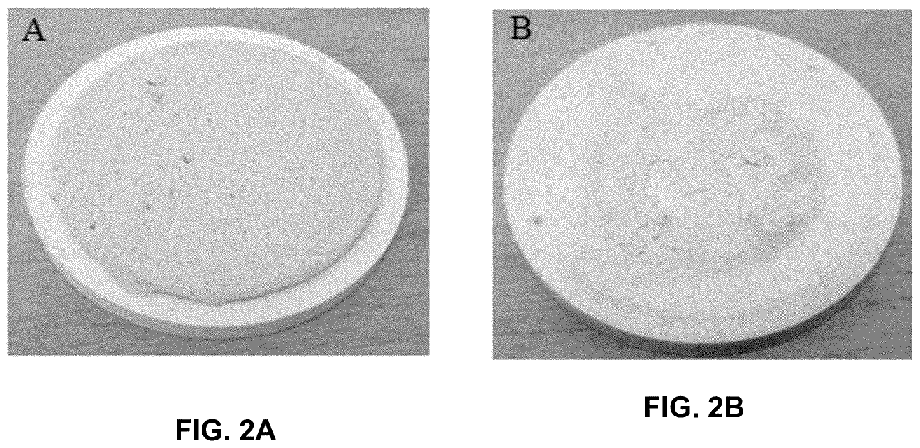

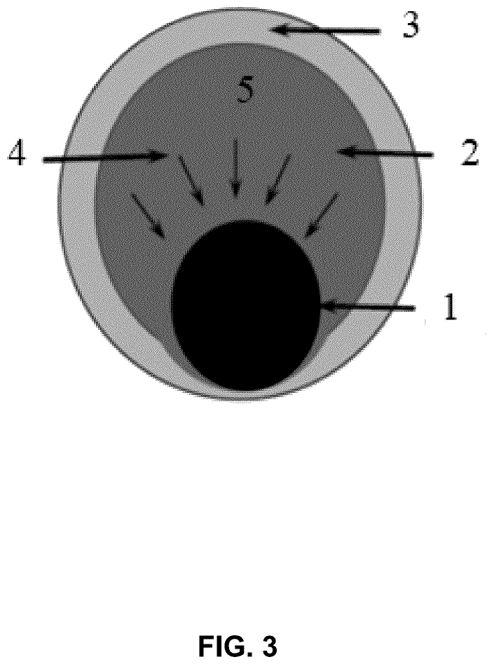

The hydrolysis/breakdown of mud cake with the stabilized enzymatic formulation (TFA 1) displayed 90% to 95% removal in 8 hours of treatment time in the laboratory setup (FIGS. 2A and 2B). This unique methodology was applied at a case in the Oil fields wherein long term stabilized enzymatic treatment fluid was pumped to release the stuck pipe in high temperature formations. The stuck pipe in the well bore is illustrated in FIG. 3. This was recorded for a newly drilled well, where Coil Tubing (CT) was run in hole tubing to the Total target depth of 11,000 feet for the objective of Well well-clean job. During running in hole, coil tubing string could not pass the depth of 9450 feet and eventually failed to be picked up after multiple efforts. The pipe could not be freed with a maximum overpull and was identified as differential stuck. On location, the conventional procedures were replaced by stable enzymatic treatment fluid composition. It was pumped across and displaced across the stuck pipe while remaining squeezed inside the well. During the 8 hours of soaking time of stabilized enzyme solution, indications were observed in the well behavior. Injectivity during pumping to free CT during the soaking period was observed to be 3.5 bpm at a well head pressure of 1300 psi. Once CT was released and stabilized enzyme fluid was pumped across at held up depth of 6000 ft as a treatment practice, the injectivity displayed an increased rate of 4.8 bpm at 1190 psi on Wellhead. This determined the successful mechanism in freeing the differentially stuck pipe as well as systematically cleaning out the filter cake over the complete accessible horizontal open hole section by the Enzyme system stabilized by the method of this invention in presence of functional cleanup fluid components. It is noteworthy that the novel stabilized formulation saved the operator more than 12 hours of non-productive time and further damage on the tubing.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Present Invention

The present disclosure provides effectively stabilizing a hydrolase enzyme in the presence of a functional cleanup fluid component. The method provides a simple way to thermally fortify and maximize the benefits of hydrolase enzymes for effective removal of biopolymer based damage from a subterranean formation.

Though enzymatic mud breaking offers a number of advantages over conventional chemical catalysts, there is every possibility of enzymes to abruptly retard or even stop working at high downhole temperatures. This makes it difficult for the operator to judge the effectiveness of enzymes in dissociating Biopolymer based formation damage, in a predictable Kinetic rate, and plan his operations accordingly. However, this method of water activity reducing agents addition to the treatment fluid will strengthen the hydrophobic interactions among non-polar amino acid residues and these interactions, together with hydrogen bonds and ionic and van der Waals interactions, would maintain the native, catalytically active structure of the enzyme and not let it be deactivated at relatively higher temperatures.

An effective enzyme hydrolysis would involve reducing the enzyme doses required to achieve the same catalytic rate or cake breakage, directly impacting the economics of the process.

Since the non-reactive nature of the enzyme treatment fluid composition, it can be easily deployed for deep well stimulation as well as fracture face damage near the surface.

Sugar, being a food grade commodity produced from natural sources, is completely environment friendly and at this level, readily biodegrades when mixed in water or land.

This method also has advantages over conventional treatments mentioned in prior works as it provides the capability of filter cake removal treatments as an additional application for freeing of stuck pipes diagnosed to be differentially stuck. The present method uses enzyme treatment fluid composition for breaking of biopolymer damage in oil and gas wells in the presence of weak organic acid by providing one step solution utilized for biopolymer-based mud filter-cake clean-up operation for new and old drilled wells and dissolution of biopolymer filer-cake build-up that produces friction against free movement of drill pipes, causing a Stuck Pipe situation. The present method delivers one stage solution for stuck pipe challenges as the enzyme holds the same properties in the presence of weak organic acid as it does during the filter cake removal. Hence it is applicable in challenging situations at high temperatures and longer stuck durations in the well.

We claim:

1. A method for stabilizing a hydrolase enzyme in the presence of a functional cleanup fluid component for breaking of biopolymer damage in an underground reservoir, or biopolymer damage buildup that causes friction against free movement of the drill pipe and differential stuck pipe situation comprising:
mixing of 2 to 60% of hydrolase enzymes to the function cleanup fluid components having 1 to 45% of organic acid buffers and esters, 3 to 20% weak organic acid or acid precursors and 1 to 5% of a fatty ethoxylate and 2 to 30% of water activity reducing agents and rest being a vehicle to obtain a stable enzyme treatment fluid composition; and
extending the enzyme treatment fluid composition into the underground reservoir via a wellbore with high downhole temperatures, using a drill string or deployed using a coiled tube or bullheaded into the fractures, while injecting the enzyme treatment fluid composition below or above the fracture pressure and shut off the contents for a period of time, or below or above the stuck pipe region where the drill pipe is differentially stuck, allowing to react and disintegrate the biopolymer based formation damage or filter cake or biopolymer caused stuck causing material completely and subsequently removed by normal flushing techniques in a known manner,
wherein the higher stability of the hydrolase enzyme of the enzyme treatment fluid composition is achieved despite the presence of the function cleanup fluid components, rendering a unique formulation that results in the stability of the enzyme protein without salting out or precipitation of the protein in presence of pH lowering organic acid,
wherein the enzyme treatment fluid composition disintegrates partially or completely the biopolymer filtercake by virtue of hydrolyzing the cross linking and matrix components of the biopolymer, also providing a reduction in the amount of force required to free said pipe.

2. The method as claimed in claim 1, wherein the underground reservoir is a hydrocarbon reservoir or water reservoir.

3. The method as claimed in claim 2, wherein the hydrocarbon is oil and/or gas.

4. The method as claimed in claim 1, wherein the hydrolase enzyme is selected from a group consisting of an alpha amylases, beta amylases, gamma amylases, cellulases, endo cellulases, exo cellulases, cellobiase, beta-glucanase, hemicellulase, mannanase, galactomannanase and combinations thereof.

5. The method as claimed in claim 4, wherein two or more hydrolase enzyme are mixed together based on the nature of biopolymer used in mud formulation.

6. The method as claimed in claim 5, where the hydrolase enzymes are mixed in the same ratio of the biopolymer substrates used in the mud formulation, on basis of declared enzyme activity units.

7. The method as claimed in claim 5, the hydrolase enzyme includes a hydrolase enzyme mix of amylases, xanthanases, cellulose enzyme and/or hemicellulase-mannanase, endoglycanases, cellobiohydrolase enzymes.

8. The method as claimed in claim 1 wherein the alpha amylase hydrolase enzyme that catalyses the endo-hydrolysis of 1,4-alpha-glycosidic linkages in starch, glycogen, and related polysaccharides and oligosaccharides containing 3 or more 1,4-alpha-linked-glucose units.

9. The method as claimed in claim 1, wherein the water activity reducing agent is a selected from a group consisting of sucrose, sorbitol, mannitol, glycerol, trehalose, propylene glycol and combinations thereof.

10. The method as claimed in claim 1, wherein the weak organic acid is selected from a group consisting of acetic acid, formic acid, citric acid, lactic acid and combinations thereof.

11. The method as claimed in claim 1, wherein the acid precursor is an ester of acetic acid, ester of formic acid, ester of citric acid, ester of lactic acid or a combination thereof.

12. The method as claimed in claim 1, wherein the organic acid buffers and ester is selected from a group consisting of chloride, carbonates, sulphate, nitrate buffers and ethanoates esters.

13. The method as claimed in claim 12, wherein the organic acid buffers and ester is selected from a group consisting of calcium chloride, sodium chloride, sodium ethanoate and combinations thereof.

14. The method as claimed in claim 1, wherein the fatty ethoxylate is selected from a group consisting of alkylphenol ethoxylates, alcohol ethoxylates, amine ethoxylates, acid ethoxylates, castor oil ethoxylates, ester ethoxylates and combinations thereof.

15. The method as claimed in claim 14, wherein the fatty ethoxylate acts as a surfactant.

16. The method as claimed in claim 1, wherein the hydrolase enzymes are a mix and are liquid enzyme formulations.

17. The method as claimed in claim 1, wherein the vehicle is selected from a group consisting of water, brine water and sea water.

18. The method as claimed in claim 1 wherein the biopolymer is selected from a group consisting of starch, xanthan, cellulose, guar derivatives and combinations thereof.

19. The method as claimed in claim 1, wherein the temperature of the formation bearing the biopolymer based filtercake or mud damage of the reservoir is at least 60° C. or higher.

20. The method as claimed in claim 1, wherein the enzyme treatment fluid composition is shut off in the reservoir for at least 45 minutes.

21. The method as claimed in claim 1, wherein the wellbore is vertical, deviated inclined or horizontal.
